(19) United States
(12) Patent Application Publication
(10) Pub. No.: US 2024/0148989 A1
(43) Pub. Date: May 9, 2024

(54) INHALER ADHERENCE MONITOR WITH MODULAR ATTACHMENTS

(71) Applicant: Reciprocal Labs Corporation, Madison, WI (US)

(72) Inventors: Samuel A. Koblenski, Madison, WI (US); Matthew Frank, Madison, WI (US); Robert Louis Baddeley, Madison, WI (US)

(21) Appl. No.: 18/506,005

(22) Filed: Nov. 9, 2023

Related U.S. Application Data
(60) Provisional application No. 63/382,987, filed on Nov. 9, 2022.

(51) Int. Cl. A61M 15/00

(52) U.S. Cl. CPC A61M 15/0083; A61M 2205/3576; A61M 2205/502

(57) ABSTRACT

An attachable adherence monitor that may be attached to different types of medicant dispensers is disclosed. The adherence monitor has a sensor body including sensing electronics that are triggered to indicate the activation of a medicant dispenser and assign a timestamp to the activation. The sensor body includes a storage device storing data indicating the activation of the medicant dispenser. The sensor body includes a connector conduit. A number of attachment modules are provided. Each of the attachment modules includes a connector that allows attachment to the sensor body via the connector conduit. A first attachment module includes a first attachment mechanism for attaching the sensor body to a first medicant dispenser. A second attachment module includes a different second attachment mechanism for attaching the sensor body to a second different medicant dispenser.

### CROSS REFERENCE TO RELATED APPLICATIONS

This present application claims the benefit of priority under 35 U.S.C. § 119(e) to U.S. Provisional Application No. 63/382,987, filed Nov. 9, 2022, the disclosure of which is hereby incorporated by reference in its entirety for all purposes.

### TECHNICAL FIELD

The present disclosure relates generally to sensors for inhalers, and more specifically for a modular inhaler adherence monitoring device that has different attachment modules to allow for use with multiple inhaler types.

### BACKGROUND

Currently, many patients with ailments are provided with an inhaler to provide dosages of drugs. For example, an asthma patient may be provided a stimulant to assist mucus and reduce inflammation by clearing up breathing passages. Thus, when the patient experiences asthma exacerbations, as a daily maintenance medication to control symptoms, the patient can put the inhaler in front of their mouth and activate the inhaler spray, delivering a dose of the drug into the lungs in order to relieve the symptom.

A known pressurized metered dose inhaler (pMDI) inhaler, such as the AstraZeneca Symbicort Rapihaler®, for inhalation of drugs, has an actuator housing at least partially defining a flow passageway extending through the inhaler from an air inlet to an outlet. A pressurized metered dose canister is held by the actuator. The canister includes a valve stem and a metering valve arranged to seat in a valve stem block formed on the housing. A main canister body of the canister may be moved relative to the housing and valve stem so as to operate the metering valve and fire a metered dose of propellant and active drug through the valve stem block and into the flow passageway. By depressing the canister, when a user inhales through a mouthpiece of the housing, air may be drawn into the housing between the canister and an inner wall of the housing, and may flow along past the canister towards the outlet. The Symbicort Rapihaler® delivers a combination of budesonide and formoterol (ICS/LABA combination) to treat asthma and/or chronic obstructive pulmonary disease (COPD). Other types of inhalers may deliver other kinds of medicaments for such ailments and other ailments. Such inhalers include the Orion Easyhaler® and the Teva Redihaler®.

One significant problem is that users often do not properly operate the inhaler, thus resulting in ineffective delivery of the medicament. For example, a user may not inhale when the medicament is dispensed from the canister and may not hold the mouthpiece in proper relation to the mouth. Unfortunately, there is no effective method to determine whether a user is following a correct technique other than health care professionals reviewing long-term health improvement after using of an inhaler for a period of time.

The use of an adherence monitor integrated into the inhaler itself has been proposed, but such a solution is expensive, and as the inhalers are designed to be disposable, such a solution is not practical. Another solution is to have an attachable monitor that may be attached to existing inhalers, such as the Symbicort Rapihaler®. However, the unique shape of the Symbicort Rapihaler® and other similar inhalers makes the physical design of such a monitor challenging.

Further, the canister of the Symbicort Rapihaler® has a label and a dose counter on the front and top that cannot be covered by the body of an adherence monitor. These features make it difficult to attach to anything but the body, and sense only on the front edge of the canister, which has the least amount of play relative to the inhaler body. The mouthpiece cover covers a substantial amount of the inhaler, and must be kept exposed. Further, the strap on the bottom of the inhaler that holds the mouthpiece cover must also be exposed. This further limits the availability of the attachment.

The nature of different physical designs for inhalers requires specially designed adherence monitors for appropriate attachment to each physical design of inhaler. This is cumbersome and presents challenges to ensure that useful data may be collected for each different inhaler design used by a patient population.

There is a need for a modular adherence monitor that may be attached to different models of inhalers to collect data related to adherence to the use of the existing inhaler. There is another need for a generic adherence monitor that may have multiple attachment component modules that may be selected depending on the type of inhaler.

### SUMMARY

One disclosed example is an attachable adherence monitor that may be attached to different types of medicant dispensers. The adherence monitor has a sensor body, including sensing electronics that are triggered to indicate the activation of a medicant dispenser and assign a timestamp to the activation. The sensor body includes a storage device, which stores data indicating the activation of the inhaler. The sensor body includes a connector conduit. A number of attachment modules are provided. Each of the attachment modules includes a connector that allows attachment to the sensor body via the connector conduit. A first attachment module includes a first attachment mechanism for attachment to a first medicant dispenser. A second attachment module includes a different second attachment mechanism for attachment to a second different medicant dispenser.

A further implementation of the example adherence monitor is an embodiment where the first attachment mechanism includes an adhesive surface for adherence to the medicant dispenser. Another implementation is where the first attachment mechanism includes a removable strip placed over the adhesive surface. Another implementation is where the first attachment mechanism includes a strap operable to be wrapped around the medicant dispenser. Another implementation is where the first attachment mechanism includes a slot holding one end of the strap. Another implementation is where the strap includes a hook and loop fastener surface. Another implementation is where the sensor body includes a cylindrical body having a bottom surface with the connector conduit. Another implementation is where the connector conduit is a plurality of semi-circular slots, each mateable to a plurality of corresponding tabs of the coupling fixture of each of the attachment modules. Another implementation is where the sensor body includes a moveable cap that may be pushed to trigger the sensing electronics. Another implementation is where the sensor body includes an auxiliary button that is operable and can be pushed to activate a function of the sensing electronics. Another implementation is where the monitor includes a battery. The sensing electronics have a low-power inventory mode, and the sensing electronics exit the inventory mode when either the cap is pushed or the auxiliary button is pushed. Another implementation is where the monitor includes a dome contact switch attached to the cap to trigger the sensing electronics. Another implementation is where the monitor includes a circuit board having the sensing electronics and dome contact switch. The circuit board is attached to the moveable cap. Another implementation is where the monitor includes an accelerometer. Another implementation is where the monitor includes a transceiver that allows communication of the stored activation data to an external device. Another implementation is where the monitor includes a visual indicator that indicates activation of the medicant dispenser. Another implementation is where the monitor includes an audio indicator that indicates activation of the medicant dispenser. Another implementation is where the first medicant dispenser is an inhaler. Lastly, another implementation is where the first medicant dispenser has a form factor, and the first attachment mechanism includes registration features matching the form factor.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1A is a perspective view of an example adherence monitor with a strap-type modular attachment component;

FIG. 1B is a perspective view of the example adherence monitor in FIG. 1A with an adhesive type modular attachment component;

FIG. 1C is a perspective view of the example adherence monitor in FIG. 1A with a specialized type of a modular attachment component;

FIG. 2A is a perspective view of the example adherence monitor in FIG. 1A;

FIG. 2B is a bottom-perspective view of the example adherence monitor in FIG. 1A;

FIG. 2C is an exploded perspective view of the components of the example adherence monitor in FIG. 1A;

FIG. 2D is a top-cutaway view of the example adherence monitor in FIG. 1A;

FIG. 2E is a side-cutaway view of the example adherence monitor in FIG. 1A;

FIG. 3A is a top view of the circuit board of the example adherence monitor in FIG. 2A;

FIG. 3B is a bottom view of the circuit board of the example adherence monitor in FIG. 2A;

FIG. 4A is a perspective view of the strap-type modular attachment component;

FIG. 4B is an exploded view of the components of the strap-type modular attachment component;

FIG. 4C is a top view of the strap component of the strap type modular attachment component;

FIG. 4D is a bottom view of the body component of the strap-type modular attachment component;

FIG. 5A is a perspective view of the adhesion-type attachment module;

FIG. 5B is an exploded view of the components of the adhesion-type modular attachment component;

FIG. 5C is a top view of the adhesion type modular attachment component;

FIG. 5D is a bottom view of the adhesion type modular attachment component;

FIG. 6A is an exploded perspective view of a customized type attachment module for the adherence monitor assembly in FIG. 1C;

FIG. 6B is a top-perspective view of the customized type attachment module in FIG. 6A attached to an inhaler;

FIG. 6C is a side-perspective view of the customized type attachment module in FIG. 6A attached to an inhaler;

FIG. 7 is a block diagram of the electronic components of the adherence monitor in FIGS. 1A-1C; and

FIG. 8 is a flow diagram of the routine that operates the adherence monitor in FIGS. 1A-1C.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to an adherence monitor attachment intended to monitor the use of different types of medicant dispensers, such as inhalers. The monitor has a sensor module that includes a sensor that may record use data from a medicant dispenser. The sensor module has a physical universal connector. The universal connector may be connected to any of a series of modular attachment components. Each modular attachment component has features that allow attachment to the universal connector of the sensor module. However, each modular attachment component has a different type of attachment mechanism that may be appropriate for one or more types of medicant dispensers. Thus, depending on the type of medicant dispenser, a user may select an appropriate modular attachment component that may be connected to the sensor module via the universal connector. The joined sensor module and modular attachment component will thus have an appropriate attachment mechanism for holding the sensor module to the type of medicant dispenser.

One example of using the adherence monitor attachment is the case of an inhaler. The user selects the appropriate modular attachment component for the inhaler, allowing the adherence monitor to be attached to the inhaler. During normal use, the user will activate the inhaler, which releases a pressurized mist of medicament. The patient either inhales the medicament directly or through an add-on spacer device. The example adherence monitor includes a switch that the user may trigger when the user activates the inhaler, and thus the adherence monitor captures a timestamp of the actuation event in on-board non-volatile (NV) memory. Other data on inhalation may be collected and added to the timestamp of the event. The adherence monitor then advertises a connection using a transmission protocol such as Bluetooth Low Energy (BLE) in order to establish a link to a client device, such as a smart phone. Once a BLE link is formed, the adherence monitor will send any event records (inhalations or heartbeats) to the client device for further analysis of adherence in relation to using the inhaler.

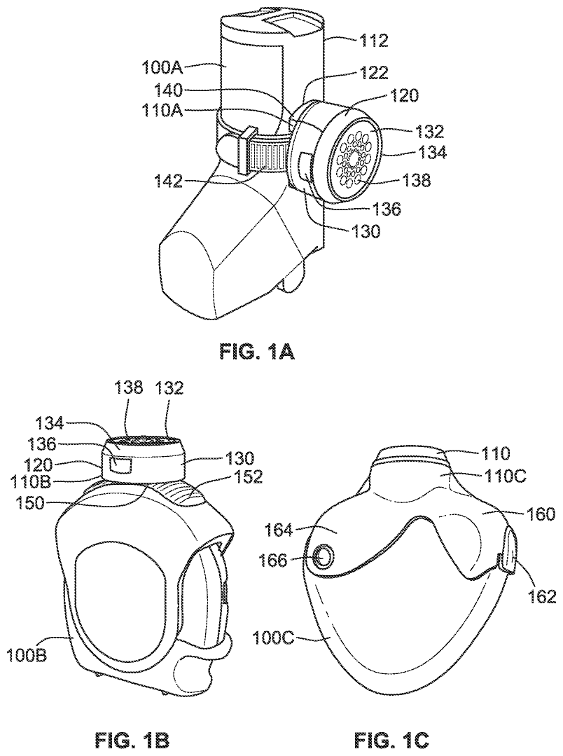

FIG. 1A is a perspective view showing an inhaler 100A mated with an example adherence monitor assembly 110A that collects data as to the firing of the inhaler 100A and other useful operational data. In this example, the inhaler 100A is a Teva Redihaler®. The adherence monitor assembly 110A has an adherence monitor module 120 that includes a main body 130, a cap 132, a retaining ring 134, and an auxiliary button 136. The cap 132 may be pushed downward relative to the main body 130 and the retaining ring 134. A top surface 138 of the cap includes perforations that allow the emission of various audio sounds from a piezo-bender.

The adherence monitor module 120 is attached to an attachment module 140 that has a uniform attachment mechanism that allows the attachment module 140 to be attached to the adherence monitor module 120. The attachment module 140 also includes a specific type of attachment mechanism that is appropriate for the inhaler 100A. The attachment module 140 includes a base component 142 and a strap component 144. In this example, the attachment mechanism is a strap attached to the strap component 144. The strap may be wrapped around the body of the Teva Redihaler® inhaler 100A to attach the adherence monitor module 120. Other similarly designed inhalers with roughly cylindrical bodies, such as such as generic pMDI inhalers with a dose counter on the canister (e.g., Cipla Albuterol generic or Bevespi Aerosphere), a Symbicort inhaler, or an Orion Easyhaler, may be used with the attachment module 140 as the strap may be wrapped around such inhalers. This allows the adherence monitor 120 to be used in these types of inhalers.

FIG. 1B is a perspective view showing an inhaler 100B mated with an example adherence monitor assembly 110B that collects data as to the firing of the inhaler 100B and other useful operational data. The assembly 110B includes the adherence monitor module 120, attached to another attachment module 150. The attachment module 150 has a uniform attachment mechanism identical to that of the attachment module 140 in FIG. 1A that allows the attachment module 150 to be attached to the adherence monitor module 120. The attachment module 150 also includes a specific type of attachment mechanism that is appropriate for the inhaler 100B. In this example, the attachment module 150 includes a base component 152 and an adhesive strip component 154. Thus, the mechanism is an adhesive surface of the adhesive strip component 154 that may be attached via an adhesive to the inhaler 100B. In this example, the inhaler 100B is a dry powder inhaler, such as an Advair Diskus dry powder inhaler that differs in design from the inhaler 100A in FIG. 1A. The inhaler 100B has several flat surfaces that allow the adhesive to attach the strip component 154. Other similarly designed inhalers with a relatively flat surface, such as other Advair generic inhalers for Seroflo Ciphaler, Stalpex DPI, and AirFluSol Forspiro, may be used with the attachment module 150. This allows the adherence monitor module 120 to be used in these types of inhalers.

FIG. 1C is a perspective view showing an inhaler 100C mated with an example adherence monitor assembly 110C that collects data as to the firing of the inhaler 100C and other useful operational data. The assembly 110C includes the adherence monitor module 120 attached to another modular attachment module 160. The modular attachment module 160 has a uniform attachment mechanism identical to that of the attachment module 140 in FIG. 1A, which allows the attachment module 160 to be attached to the adherence monitor module 120. The attachment module 160 also includes a specifically designed attachment mechanism that includes specific registration features for attachment to the inhaler 100C. The attachment module 160 thus includes a front registration feature 162 and two parallel sections 164 that include a hole 166 that may be fit over a post or axle on a rotatable mouthpiece cover of the inhaler 100C. In this example, the inhaler 100C is a Wixela Inhub dry powder inhaler that has a different form factor than that of either the inhalers 100B in FIG. 1B or 100A in FIG. 1A.

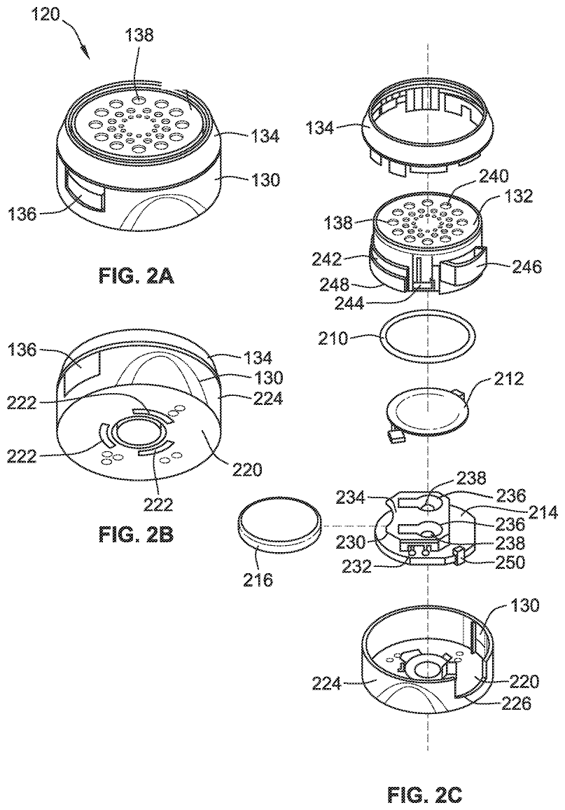

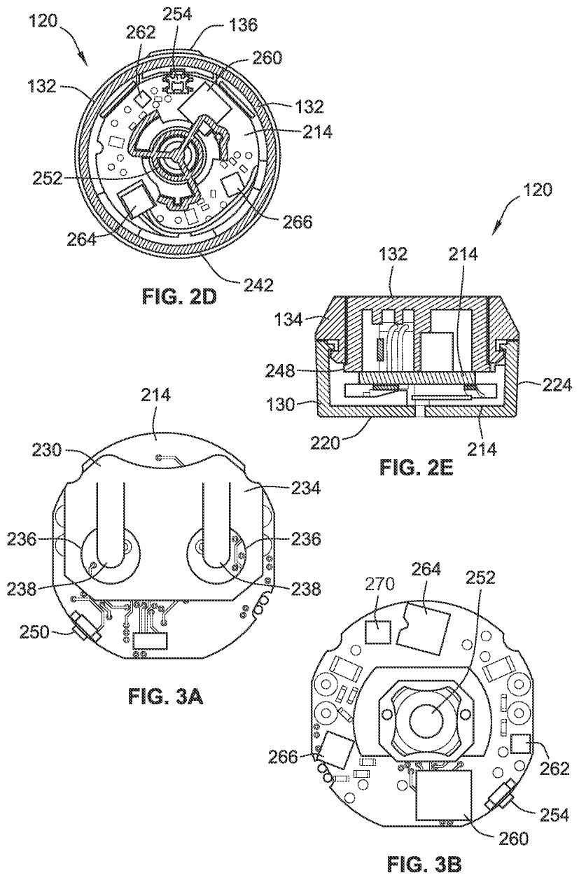

The adherence monitor assemblies 110A, 110B, and 110C are respectively attached to the inhalers 100A, 100B, and 100C in FIGS. 1A-1C. Each of the adherence monitor assemblies has the adherence monitor module 120 that is shown in FIGS. 2A-2E. Thus, FIG. 2A is a top perspective view of the adherence monitor module 120; FIG. 2B is a bottom perspective view of the adherence monitor module 120; FIG. 2C is an exploded perspective view of the components of the adherence monitor module 120; FIG. 2D is a top cutaway view of the adherence monitor 120; and FIG. 2E is a side cutaway view of the adherence monitor module 120.

As shown in FIGS. 2A-2C, the retaining ring 134 holds the moveable cap 132 in place relative to the main body 130. A ring of RTV adhesive 210 is applied to a circular channel inside the cap 132. The adhesive ring 210 holds a circular plate piezo-bender 212 in place and retains flexibility so the piezo-bender plate 212 can vibrate. The main body 130 is cylindrically shaped with an open end and an opposite closed end 220. The retaining ring 134 has interior tabs that may be rotated into slots on the interior walls of the main body 130 to fix the retaining ring 134 in place relative to the main body 130 to hold the moveable cap 132 in the main body 130. A circular circuit board 214 is attached to the open end of the moveable cap 132. The piezo-bender 212 is suspended above the circular circuit board 214 in the interior of the moveable cap 132. A battery 216 is inserted over the circuit board 214 and held in place by a battery holder structure 230.

As shown in FIG. 2B, the bottom surface of the bottom end 220 has a pattern of three semi-circular slots 222 that form a universal attachment mechanism. The semi-circular slots 222 are mated with corresponding tabs in any of the modular attachment components. After the tabs are mated with the slots 222 in the main body 130, the main body 130 is rotated a quarter turn, and small ridges on the tabs align with indentations on the slots 222 to lock the main body 130 to the attachment component. A rotation with sufficient force in the opposite direction will overcome the friction lock and allow the attachment component to be removed from the main body 130. A cylindrical wall 224 is joined to the top of the circular bottom end 220. The cylindrical wall 224 includes a cutout 226, where the auxiliary button 136 is seated.

The battery holder structure 230 is suspended above the circuit board 214. The support structure 230 has two support members 232 and a top panel 234 with two apertures 236. The battery 216 is inserted between the two support members 232 and under the top panel 234. A pair of flexible positive contacts 238 are moveable within matching apertures 236 to contact the battery 216. A negative contact on the circuit board 214 contacts the opposite side of the battery 216 and closes the power circuit.

The cap 132 includes the closed top surface 138 with indentations 240. The top surface 138 is joined to a cylindrical wall 242. The cylindrical wall 242 includes a series of slots 244 that allows the circuit board 214 to be supported near the open end of the cap 132. A button mounting tab 246 is shaped to hold the auxiliary button 136. A protrusion on the button tab 246 is shaped to be an easily pressed surface for the user. When the surface of the auxiliary button 136 is pressed, the bottom edge of the button tab 246 presses a switch on the circuit board 214. The mounting tab 246 is aligned in the cutout 226 when the cap 132 is inserted into the main body 130. An annular bottom collar 248 extends from the cylindrical wall 242. The annular collar 248 has a wider diameter than the retaining ring 134, and thus the retaining ring 134 holds the cap 132 within the main body 130.

The adherence monitor module 120 is powered by the battery 216. In this example, the battery 216 is a single non-rechargeable (primary) coin cell type battery, such as a CR2032 3V coin cell battery. The intended battery life is 3 years of shelf life followed by 1 year of use life, with no intention for the average user to be able to replace the battery. An extra-low power inventory mode is used to preserve the battery charge during the shelf-life period. Of course, rechargeable batteries may be used, or other sources of power may be used.

FIG. 3A is a top view of the circuit board 214, and FIG. 3B is a bottom view of the circuit board 214. Both sides of the circuit board 214 with electronic components attached by soldering or other attachment mechanisms. The top side of the circuit board 214 supports an LED 250 and the support structure 230.

As shown in FIG. 2D and FIG. 3B, the bottom side of the circuit board 214 supports a snap dome switch 252 that, when depressed, provides electrical connections between contacts on the bottom side of the circuit board 214. An auxiliary switch 254 is mechanically coupled to the button tab 246 and auxiliary button 136. Pressing the auxiliary button 136 thus moves the button tab 246 and activates the auxiliary switch 254. The bottom side of the circuit board 214 also includes a microprocessor chip 260, a piezo driver chip 262, a piezo driver socket 264, and an accelerometer 266. In this example, the microprocessor chip 260 is a BGM220 ARM Cortex-M33 with a built-in BLE radio. Of course, any suitable microprocessor or controller may be used with an integrated radio. Alternatively, the radio may be in a separate chip. The microprocessor chip 260 also includes on-chip non-volatile memory. The accelerometer 266 in this example, is a LIS2DH very low-power 3-axis accelerometer. The accelerometer 266 provides orientation data at the moment the primary button is pressed. If the location of the monitor module 120 on the medication is known, then the orientation can provide insight on how the medication was taken. The accelerometer 266 also detects when the monitor module 120 is shaken, which can be included as extra data on how the medication was taken when the primary button is pressed, or can directly detect usage of some types of nebulizers, including duration of use.

A snap dome switch 252 is designed to target a specific trip force. The snap dome switch 252 triggers the sensor for recording use of the respective inhaler when the primary button in the form of the cap 132 is pressed down relative to the retaining ring 134. The force required to trip the snap dome switch 252 is appropriate for the size of the cap 132, so that it will be comfortable for the user when pressing down the cap 132.

The auxiliary button 136 is located on the side of the body 130. The auxiliary button 136 has multiple functions including toggling reminder sounds on/off and triggering heartbeat events. The auxiliary button 136 is connected to the cap 132. In this example, the auxiliary button 136 is a translucent acetal material and acts as the actuator for the auxiliary switch 254 and the light pipe for the LED 250.

The green LED 250 is visible through the face of the auxiliary button 136 on the side of the main body 130. In this example, the LED 250 is used with various flash/strobe combinations to provide the user with feedback about the status of the operation of the adherence monitor module 120. In this example, the LED 250 is controlled by a single GPIO pin of the microprocessor 260.

The adherence monitor module 120 incorporates the piezo-bender 212 to give the user audible feedback. The piezo-bender 212 is used to provide various tones and indications (i.e., button push feedback, reminder tones, etc.) provided by the piezo-bender driver component 264. The driver component 264 is activated by a signal from the microprocessor 260. In this example the piezo-bender driver component 264 is driven by three GPIO pins of the microprocessor 260 that include one PWM signal and two volume control signals. The piezo-bender 212 is directly powered by the piezo driver chip 262 through two wires from the socket 264. Alternatively, the piezo-bender 212 may be powered directly from the microprocessor with a pair of inverted PWM signals. The circuit board 214 has an option to direct-drive if the piezo-bender driver chip 262 is not used.

In other embodiments, the adherence monitor module 120 comprises a microphone 270. In one aspect, the microphone can comprise MMICT3902 from TDK Invensense type microphone. Similar to the dome switch 252 and the accelerometer 266, the microphone can provide additional usage data for the user. In a further aspect, the microphone can be configured to work in a low power or power-saving mode, such that the microphone can be triggered in response to the activation of the dome switch 252 or accelerometer. During operation, the microphone 270 can be in an initially dormant state, the microphone can then be actuated into an active state by the adherence monitor module 120. In another example, the user can be initiating medication use from an inhaler, the accelerometer 266 can be activated when the inhaler is moved to the patient's mouth. The movement of the accelerometer 266 can initiate monitoring by the microphone wherein the patient's breathing pattern can be detected and analyzed for abnormalities or consistency with a healthy patient. The analysis by the adherence monitor 120 to identify abnormalities or healthy breathing can be performed by performing a signal analysis on the audio signal received from the microphone 270.

In one aspect, the audio signal analysis can perform a Fast Fourier Transform (FFT) to match a desired breathing profile, where the profile is the characteristic shape of the frequency response of the FFT on the frequency spectrum. For example, regular inhalation can be depicted with in the frequency domain with peaks located along the frequency axis. For example, a healthy inhale or exhale may produce a representation showing a peak at 400 Hz. Any received from data from the breathing activity of the user can be compared to determine the divergence from the representation of the strong inhale or exhale at 400 Hz. In a further aspect, the entirety of the representative breathing profile can be evaluated wherein peaks at other frequencies for a regular inhalation are compared to the data received. Similarly, frequency profiles characterized as weak inhalation can be compared with the received data to further characterize the breathing activity. In a further aspect, the analysis can identify and filter noise to generate a more accurate profile representation.

Regarding the various sensors, including the dome switch 252, accelerometer 266, and microphone 270, a plurality of data can be acquired to determine additional usage parameters associated with medication events for the patient. For example, the dome switch 252 can detect the duration of the press and the timestamp at which the switch is actuated. With the accelerometer 266 and microphone 270 can attach additional information, regardless of whether each is used for detection. For example, when the dome switch 252 is pressed, the accelerometer 266 reading can be captured at that time and combined with the orientation data of the sensor at the time of actuating the done switch. Further, when sensed vibrations initiate data capture, the system can also capture the duration of the event. When audio profile detection for a medication event is initiated after 1) pressing the dome switch or 2) sensing vibration from the accelerometer, data capture parameters can include: peak air flow, inhalation duration, time to peak (from the start of inhalation), total volume, and others. In the case of a metered dose inhaler where this sensor is attached to the top of the canister, an initiation of data capture can comprise a manual button actuation. Further, the accelerometer 266 can be used to detect the orientation of the inhaler at the time of medication dispensing, as well as, whether the patient shook their inhaler first.

Power management capabilities associated with the microphone 270, can be maintained via automatic operation or manual operation. The power management capabilities can be configured to operate the microphone 270 in a low-power mode. The microphone 270 can be directly actuated by pressing the dome switch 252, which is in electronic communication with the microphone. In another aspect, the adherence monitor module 120 can comprise a timing protocol. For example, adherence monitor module 120 can deactivate the microphone when audio signals below a certain decibel threshold are not detected for a predetermined period of time. Further, the threshold decibel level can be adjusted by the adherence monitor module by continuously feeding audio data into the machine learning model or neural net to determine if the audio signal received is background noise or audio activity generated by the patient. In an aspect, the machine learning model or neural net can comprise an FFT algorithm. For example, the machine learning could be used to sample a variety of known good inhales and known bad inhales, and running the algorithm to generate a neural net that is capable of distinguishing between the two. In a further aspect, this microphone shutoff protocol could also include the data received from the accelerometer 266 to further enhancing the timing for microphone shutoff.

The adherence monitor module 120 monitors the activation of any type of medicant dispenser. One type of medicant is inhaler-based medication, such as those for Asthma and COPD. However, other types of medicants and dispensers may use the adherence monitor module 120 to record activation with the appropriate attachment module. In this example, the different attachment modules 140, 150, and 160 in FIGS. 1A-1C allow the use of the adherence monitor module 120 for different inhaler form factors for which a custom adherence monitor is unavailable. The sensor assembly of the adherence monitor 120 and appropriate attachment module allow attachment to an appropriate area of any medication container or delivery device, such as pill bottles, blister packs, nebulizers, nasal sprays, and topical cream containers. Before or after normal use of a medicant dispenser, the user will press down on the cap 132 of the adherence monitor module 120 to indicate use of the medicant dispenser. In response to the cap 132 being pressed, the adherence monitor module 120 captures a timestamp of the medication usage event in the on chip nonvolatile memory of the microprocessor chip 260. The adherence monitor module 120 then advertises a connection using a wireless communication protocol such as BLE in order to establish a link to a client device. Once a BLE link is formed, the adherence monitor module 120 will send any event records (inhalations or heartbeats) to the client device.

The adherence monitor module 120 may be secured to the variety of attachment modules using a bottom surface with a three-pronged twisting interlock mechanism with the slots 222 shown in FIG. 2B. As will be explained, any of the attachment modules, such as the modules 140, 150, and 160 have mating tab features that may be inserted on the slots 222 of the interlock mechanism. The attachment module is then twisted to lock the tabs into corresponding locking slots 222 to hold the attachment module to the adherence monitor module 120.

Attachment modules can be designed with different mechanisms, such as adhesives, hook and loop fasteners, straps, medication-specific collars, or other devices for different attachment modules, such as the modules 140, 150, and 160 in FIGS. 1A-1C. The attachment modules are molded separately from the rest of the components of the adherence monitor module 120 to allow them to be expanded upon with new options or tailored to a specific medication delivery device, such as the example in FIG. 1C. Thus, the adherence monitor module 120 can be transferred to a new medicant dispenser once the medicament has been spent by removing the attachment accessory from the old medicant dispenser. If the new medication delivery device is incompatible with the attachment module, a more appropriate attachment module may be selected and joined with the adherence monitor module 120.

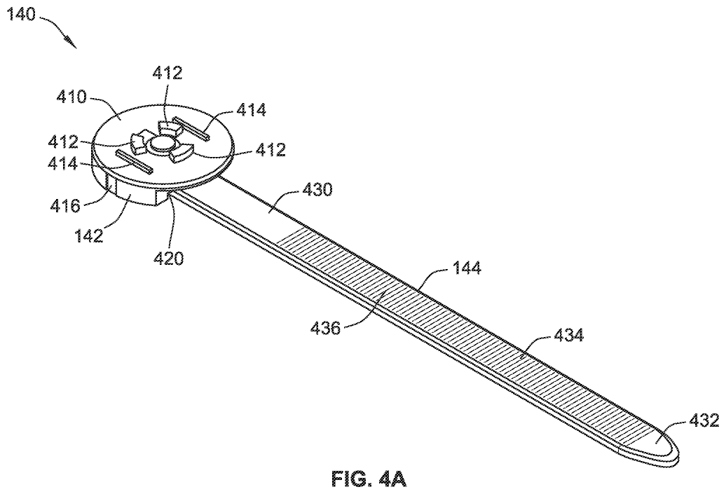

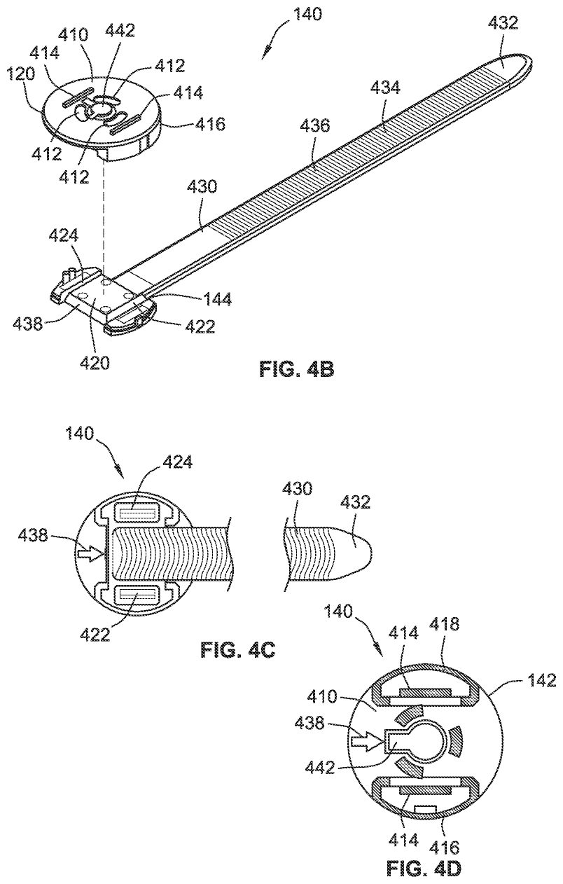

FIG. 4A is a perspective view of the strap-type modular attachment module 140 in FIG. 1A. FIG. 4B is an exploded view of the base component 142 and the strap component 144 of the strap-type modular attachment module 140. FIG. 4C is a top view of the base component 142 and the strap component 144 of the strap type modular attachment module 140. FIG. 4D is a bottom view of the base component 142 of the strap-type modular attachment module 140.

The base 142 component is generally cylindrical with a top surface 410. The top surface 410 includes three registration tabs 412 (coupling fixtures) that may mate to the slots 222 (connector conduit) of the monitor module 120. The top surface 410 also includes two parallel slots 414 cut through the base component 142. Two side sections 416 and 418 extend from the top surface 410 to allow attachment of the strap component 144.

As shown in FIGS. 4A-4B, the strap component 144 includes a main section 420 that includes two snap hooks 422 and 424 that may be inserted in the slots 414 of the base component 142 to hold the main section 420 to the base component 142. The main section 420 supports a strap 430 that is of sufficient length to encircle the body of a medication device.

The strap 430 includes a triangular attachment end 432. The strap 430 includes a top surface 434 that has a series of ridges 436. The ridges 436 allow a friction lock to a back slot 438 formed by the main section 420 being joined to the base component 440. The two side sections 416 and 418 extend from the top surface 410, and create the slot 438 between the base component 142 and the strap component 144 for the triangular end 432 of the strap 430 to feed through.

The strap 430 is flexible and may be wrapped around an inhaler body. The triangular end 432 is inserted into the back slot 438 and pulled through the slot 438 to tighten the strap 430. Attaching the adherence monitor module 120 causes a center lever-type locking mechanism 442 to be depressed. A protrusion on the underside of the locking mechanism 442 engages with the ridges 436, holding the strap 430 in place. When the adherence monitor module 120 is attached, the body of the module 120 presses down the center of the locking mechanism 442 so that a tooth on the underside presses into the strap ridges 436. The protrusion and ridges 436 have a sloped surface in one direction to allow the strap 430 to be tightened in a ratcheting method. The strap 430 can only be loosened by removing the adherence monitor module 120 from the attachment module 140 to release the locking mechanism 442 and detach the protrusion from the ridges 436. Alternatively, the strap 430 may have one surface of a hook and loop fastener and thus may be attached to itself and held by the hook and loop fastener.

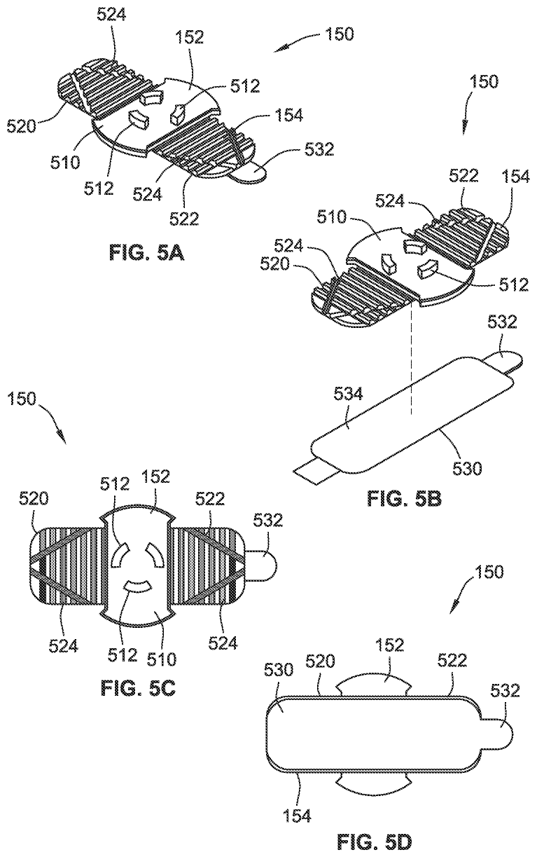

FIG. 5A is a perspective view of the adhesive type of attachment module 150. FIG. 5B is an exploded view of the base 152 and the adhesive support component 154 of the adhesive type of modular attachment component 150. FIG. 5C is a top view of the base component 152 and the adhesive component 154 of the adhesive type of modular attachment component 150. FIG. 5D is a bottom view of the base component 152 and the adhesive component 154 of the strap type modular attachment module 150.

The base component 152 has a top surface 510 that includes three semi-circular registration tabs 512. The tabs 512 allow attachment of the base 150 to the slots 222 of body 130 of the adherence monitor module 120 in FIG. 2B. The adhesive support component 154 has two extending wing sections 520 and 522. Each of the wing sections 420 and 424 are composed of a series of parallel lateral ribs 524. The lateral ribs 524 allow both wing sections 520 and 522 to be bent to conform to a curved surface. An adhesive is applied to the bottom of the wing sections 520 and 522. As shown in FIG. 5B-5C, a protective strip 530 is initially attached to the bottom surfaces of the wing sections 520 and 522 to protect the adhesive. The protective strip 530 has a tab 532 that allows a user to remove the protective strip 530 when the attachment module 150 is ready to be attached via the adhesive on the wing sections 520 and 522. The protective strip 530 has a non-stick surface 534 that is joined to the adhesive on the bottom surfaces of the wing sections 520 and 522. An opposite surface of the protective strip 530 is a non-adhesive base layer.

After the attachment module 150 is joined to the adherence module 120, a user may remove the protective strip 530 to expose the bottom surfaces of the wing sections 520 and 522 with the adhesive. The protective strip 530 may then be discarded. The wing sections 520 and 522 may be bent in order to conform with the surface of an inhaler. The bottom surface of the wing sections 520 and 522 are placed in contact with the surface of the inhaler. The adhesive thus bonds the attachment module 150 to the inhaler.

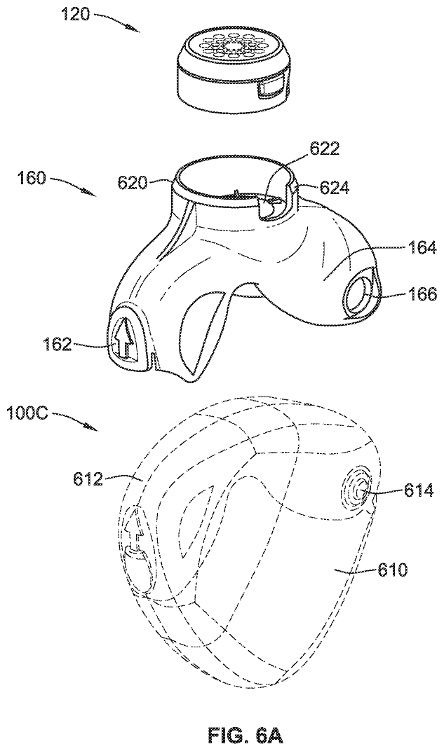

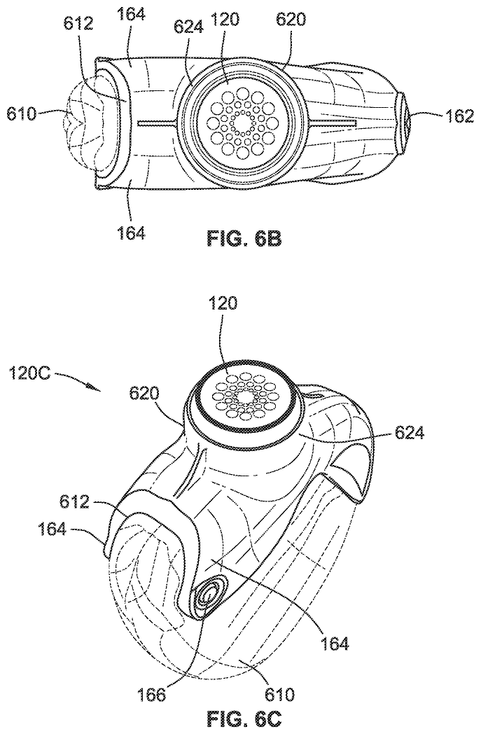

FIG. 6A is an exploded perspective view of the customized type of attachment module 160 for the adherence monitor assembly 110C attached to the inhaler 100C. FIG. 6B is a top-perspective view of the customized-type attachment module 160 attached to the inhaler 100C. FIG. 6C is a side-perspective view of the customized type of attachment module 160 attached to the inhaler 100C.

As explained above, the inhaler 100C in this example is a dry powder inhaler that includes a roughly trapezoidal shaped body 610 and a rotatable mouthpiece cover 612. The cover 612 is rotatable and mounted on a pair of pins 614. When, the inhaler 100C is used, the user rotates the cover 612 away from the body 610, thus opening an outlet for the stored medication.

In this example, the adherence monitor assembly 110C includes the adherence module 120 that is attached to the specialized attachment module 160. The attachment module 160 includes a cylindrical mounting structure 620. The mounting structure 620 has a bottom panel 622 that is defined by a circular wall 624. The adherence module 120 is inserted in the mounting structure 620. The slots at the bottom of the adherence module 120 are mated to tabs similar to those in the other attachment modules.

The cylindrical mounting structure 620 is molded as part of the entire attachment module 160. The remainder of the attachment module 160 is shaped to cover the mouthpiece cover 612. Thus the holes 166 are aligned with the pins 614. The sections 164 are attached to the other end of the mouthpiece cover 612. Thus, the attachment module 160 moves with the mouthpiece cover 612. The specialized attachment module 160 is designed for a specific inhaler form factor. Other different modules may be designed for mating with other specific types of inhaler form factors.

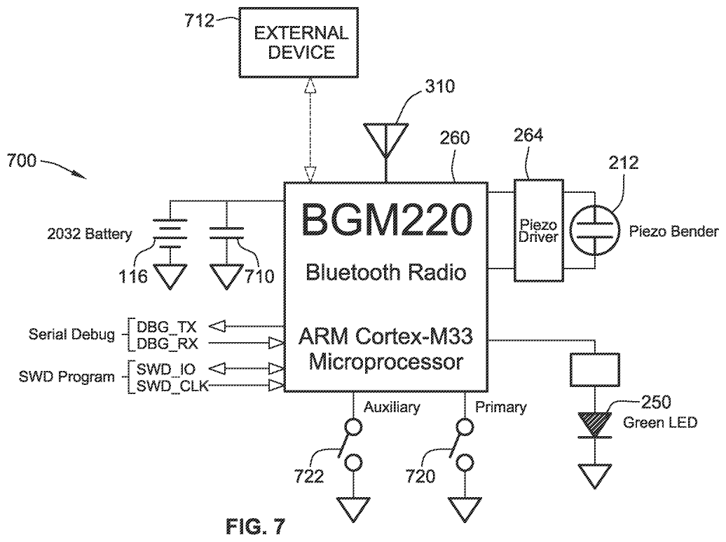

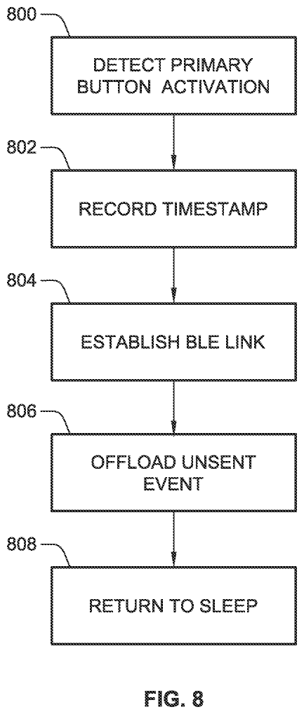

FIG. 7 is a block diagram of the sensor electronics 700 of the adherence module 120. The battery 216 provides DC power through a smoothing capacitor 710. In this example, the microprocessor 260 includes a DC-DC switching regulator. The built-in BLE radio in the microprocessor 260 transmits and receives signals through the on-board antenna 310. The signals may be exchanged with an external device 312 such as a smartphone or a Cloud-based device. As explained above, the microprocessor 260 includes GPIO outputs for the piezo-bender driver 264 for driving the piezo-bender 212. The microprocessor 260 thus may generate audio signals through driving the piezo-bender 212. Visual indications may be generated via the microprocessor 260 sending signals to light the LED 250.

As explained above, a primary switch 720 is activated by pressing down the cap 132 to provide electrical contact via the contact dome 252. An auxiliary switch 722 is part of the switching circuit 254 and is activated by pressing the auxiliary button 136. Both of the switches 720 and 724 provide input signals to the microprocessor 260.

The firmware executed by the microprocessor 260 enables a series of functions activated by pressing the cap 132 or the auxiliary button 136. Pressing the auxiliary button 136 once adds a manual heartbeat to the event queue and the microprocessor will attempt to connect to the client. The adherence monitor module 120 is shipped in inventory mode, which keeps the components in the adherence monitor module 120 at the lowest possible sleep modes. Pressing either the auxiliary button 136 or pressing the cap 132 once will remove the sensor from inventory mode. Once exiting inventory mode, the routine will register a usage when the cap 132 is pushed to activate the dome switch 252. In a silent mode, three consecutive pushes of the auxiliary button 136 turns reminder tones off. Two consecutive pushes of the auxiliary button 136 turns reminder tones on. A reminder schedule of up to four times per day of the week can be set in the microprocessor flash memory by sending commands over a BLE connection. A built-in real-time clock (RTC) is monitored, and if the RTC reaches a scheduled reminder time without a primary button press recorded, a reminder tone will play on the piezo-bender 212. After a programmable delay, the reminder tone will play up to two more times, unless the primary button is pressed to indicate a dose of medication has been taken. A push of the auxiliary button 136 during a reminder tone will silence that reminder sequence for any given scheduled dose.

The client 712 updates the on-sensor reminder schedule via a parameter update. The parameter update is sent from the client device 712 after all events have been successfully offloaded from the adherence monitor module 120. The schedule is set by the patient via a web application that may be executed by the client device 712. When the scheduled time occurs, the microprocessor 260 activates the piezo-bender 212 with a customized sequence of frequencies.

For most of its life, the adherence module 120 will be in sleep mode. At rest, the primary button contact dome switch will be open. When a user presses down on the primary button, the force of the press overcomes the trip force of the snap dome. When the snap dome trip force is reached, the snap dome shorts the circuit, and the microcontroller will be interrupted with a primary button push event. The sensor will wake up and record a timestamp associated with the RTC, and store an event in the event queue.

Once the microprocessor 260 detects that the cap 132 has been pressed and stores the information in the event queue, the microprocessor 260 attempts to offload the events in the queue to the external client device 712. This transmission takes place via the BLE wireless protocol implemented inside the microprocessor 260. In this example, communication between the ARM Cortex-M33 processor and the BLE stack happens internally to the microprocessor 260. The client device 712 indicates the success or failure of the event transmission, which is then communicated back to the firmware running on the ARM Cortex-M33.

FIG. 8 is a flow diagram of the routine executed by the microprocessor 260 to record the actuation events of the inhaler 10. The flow diagram in FIG. 8 is representative of example machine-readable instructions for collecting and analyzing adherence data collected from the adherence monitor 100 in FIG. 1B. In this example, the machine-readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application-specific integrated circuit [ASIC], a programmable logic device [PLD], a field-programmable logic device [FPLD], a field-programmable gate array [FPGA], discrete logic, etc.). For example, any or all of the components of the interfaces can be implemented by software, hardware, and/or firmware. Also, some or all of the machine-readable instructions represented by the flowcharts may be implemented manually. Further, although the example algorithm is described with reference to the flowchart illustrated in FIG. 8, persons of ordinary skill in the art will readily appreciate that many other methods of implementing the example machine-readable instructions may alternatively be used. For example, the order of execution of the blocks may be changed, and/or some of the blocks described may be changed, eliminated, or combined.

FIG. 8 shows a flow diagram of the routine operated by the microprocessor 260. The routine is initiated when the microprocessor detects that the primary button has been pushed (the snap dome has been tripped) (810). In an another aspect, the microprocessor may be activated when an audio signal is detected from the microphone or movement is detected from the accelerometer. The processor records the timestamp of the primary button push (802). A Bluetooth link is formed with the client external device (804). The BLE radio on the microprocessor 260 offloads unsent events in the queue (806). The microprocessor goes back to its sleep state once events have been sent (808). In a further aspect, the microphone can receive audio signals, wherein the processor is activated. The processor can perform a FFT analysis by converting the electrical signals received from the microphone generated from the breathing and/or heartbeat activity of the user. The processor can make a comparison between the received signal and previously stored data to make a profile analysis. The resultant comparison exceeding a threshold an initiate alert that can be saved and transmitted.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform a specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon reading and understanding this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

What is claimed is:

1. An attachable adherence monitor, the adherence monitor being attachable to a plurality of different types of medicant dispensers, the adherence monitor comprising:
a sensor body including: a processor in communication with sensing electronics that are triggered to indicate the activation of a medicant dispenser and assign a timestamp to the activation, a storage device in communication with the processor further configured to store data indicating activation of the medicant dispenser, and a connector conduit; and
an attachment module including a coupling fixture that allows attachment to the sensor body via the connector conduit and a first attachment mechanism for attaching the sensor body to a first medicant dispenser.

2. The adherence monitor of claim 1, wherein in the storage device is further configured to store a plurality of breathing profiles, wherein each breathing profile comprises a frequency-spectrum representation of a user breathing activity.

3. The adherence monitor of claim 2, further comprising a microphone being configured to generate electrical signals based on audio signals received during usage of the medicant dispenser, such that the electrical signal are transformed to the frequency spectrum representation for comparison with the plurality of breathing profiles.

4. The adherence monitor of claim 1, wherein a second attachment module includes a second attachment mechanism for attaching the sensor body to second medicant dispenser, wherein the first and second attachment mechanisms are different and wherein the first and second medicant dispensers are different.

5. The adherence monitor of claim 1, wherein the first attachment mechanism includes an adhesive surface and a removable strip placed over the adhesive surface for adherence to the medicant dispenser.

6. The adherence monitor of claim 1, wherein the first attachment mechanism includes a strap operable to be wrapped around the medicant dispenser.

7. The adherence monitor of claim 6, wherein the first attachment mechanism includes a slot holding one end of the strap.

8. The adherence monitor of claim 6, wherein the strap includes a hook and loop fastener surface.

9. The adherence monitor of claim 1, wherein the sensor body includes a cylindrical body having a bottom surface defining the connector conduit.

10. The adherence monitor of claim 1, wherein the coupling fixture comprises a tab configured to engages the connector conduit comprising at least one semi-circular slot.

11. The adherence monitor of claim 1, wherein the sensor body includes a moveable cap that is operable to activate the sensing electronics.

12. The adherence monitor of claim 11, further comprising an auxiliary button that is operable to activate the sensing electronics.

13. The adherence monitor of claim 12, further comprising a battery, wherein the sensing electronics have a low power inventory mode, and wherein the sensing electronics exit the inventory mode when either the cap is pushed or the auxiliary button is pushed.

14. The adherence monitor of claim 11, further comprising a dome contact switch attached to the cap to trigger the sensing electronics.

15. The adherence monitor of claim 11, further comprising a circuit board holding the sensing electronics and dome contact switch, wherein the circuit board is attached to the moveable cap.

16. The adherence monitor of claim 1, further comprising an accelerometer.

17. The adherence monitor of claim 1, further comprising a transceiver that allows communication of the stored activation data to an external device.

18. The adherence monitor of claim 1, further comprising at least one of a visual indicator or audio indicator that indicates activation of the medicant dispenser.

19. The adherence monitor of claim 1, wherein the first medicant dispenser is an inhaler.

20. The adherence monitor of claim 1, wherein the first medicant dispenser has a form factor, wherein the first attachment mechanism includes registration features matching the form factor.
